(19) United States
(12) Patent Application Publication
(10) Pub. No.: US 2002/0095260 A1
(43) Pub. Date: Jul. 18, 2002

(54) Methods for efficiently mining broad data sets for biological markers

(75) Inventor: Nam Q. Huyn, Milpitas, CA (US)

Correspondence Address: SWANSON & BRATSCHUN L.L.C., 1745 SHEA CENTER DRIVE, SUITE 330, HIGHLANDS RANCH, CO, 80129, US

(73) Assignee: SURROMED, INC.

(21) Appl. No.: 09/994,576

(22) Filed: Nov. 27, 2001

Related U.S. Application Data
(60) Provisional application No. 60/253,656, filed on Nov. 28, 2000.
(60) Provisional application No. 60/271,091, filed on Feb. 23, 2001.

(51) Int. Cl. G06F 7/00; G06F 17/30; G06F 19/00; G01N 33/48; G01N 33/50

(52) U.S. Cl. 702/19; 707/3

(57) ABSTRACT

A biological marker identification method identifies biological markers within broad sets of biological data containing many more measurements than observations. For example, the data can contain thousands of measurements on each blood sample obtained from fewer than 100 subjects, each of which falls into one of a set of clinical classes or is associated with a value of a continuous clinical response variable. At least one biomarker, containing a small subset of measurements, is found that is capable of predicting a clinical endpoint. The biomarker can be used for, e.g., diagnosing disease or assessing response to a drug. First, the set of measurements is reduced to a smaller set of candidate measurements by eliminating measurements that either cannot distinguish among classes or are redundant. Biomarker subsets are then selected from the remaining set of measurements, either by an exhaustive search or a heuristic method that finds good but not necessary globally optimal biomarkers.

### CROSS-REFERENCE TO RELATED APPLICATIONS

This application claims the benefit of U.S. Provisional Application No. 60/253,656, “Methods for Efficiently Mining Broad Data Sets for Biological Markers,” filed Nov. 28, 2000; and U.S. Provisional Application No. 60/271,091, “Data Analysis and Mining in the Life Sciences,” filed Feb. 23, 2001, both of which are herein incorporated by reference.

### FIELD OF THE INVENTION

The present invention relates generally to analysis of biological data. More particularly, it relates to methods for mining broad data sets of biological measurements to identify subsets of measurements that are predictive of clinical endpoints such as clinical classifications (e.g., disease conditions or responses to drug therapy) or continuous clinical response variables (e.g., degree of disease progression).

### BACKGROUND OF THE INVENTION

An important goal of a growing number of biological researchers is to discover and identify novel biological markers. A biological marker, or biomarker, is a characteristic that is measured and evaluated as an indication of normal biological processes, pathogenic processes, or pharmacological responses to therapeutic intervention. New biomarkers are being sought to enable diseases to be diagnosed more accurately or earlier than is currently possible. Responses to drug therapy can also be gauged earlier and more accurately using biomarkers, promising to accelerate the progress and reduce the cost of clinical trials. Biomarker discovery is concentrated primarily on chronic diseases for which many of the complex pathogenic mechanisms are still unknown, such as Alzheimer's disease, rheumatoid arthritis, and diabetes.

Although a variety of approaches are possible for biomarker discovery, one of the most promising is the so-called shotgun approach, in which enormous volumes of biological measurements are acquired from different classes of subjects and then mined to identify biomarkers capable of distinguishing among the subject classes or otherwise predicting clinical endpoints. The philosophy behind this approach is that any type of measurement may be important to a particular disease, and so measurements should not be constrained to those known to be relevant. The shotgun approach has been made possible in recent years through advances in high-throughput measurement technologies such as gene chips, protein chips, and mass spectrometry. These tools are capable of detecting hundreds of thousands of proteins and small organic molecules within tiny volumes of biological materials, resulting in high volumes of measurement data. In fact, the current bottleneck in biomarker discovery is not in obtaining varied biological data, but in managing and analyzing the generated data.

One of the problems is that data mining techniques developed for financial or commercial applications are not directly applicable to the biotechnology domain. Because of the context in which they are acquired, biological measurement data are fundamentally different from other data types. Biomarker discovery is commonly performed on data gathered from clinical studies investigating a particular condition or set of conditions or a particular drug treatment. Studies are described by a well-characterized collection of subjects, a particular sample type (e.g., blood) and conditions for sample acquisition, and specific measurement methods. The table of FIG. 1 illustrates the conceptual structure of an example data set acquired from a clinical study. Rows of the table correspond to observations, each identified by an observation number. Each observation refers to, for example, a sample taken at a particular time from a patient belonging to one of a predetermined set of clinical classes. Alternatively, an observation can refer to a single patient from whom single or multiple samples are taken. Associated with each sample or observation is a large number of biological measurements, indicated by the m₁ columns of the table of FIG. 1. Examples of measurements are concentration of a soluble factor in the blood, blood cell population, intensity of a mass spectral peak obtained after subjecting the sample to mass spectrometry, or lifestyle factor such as smoking or amount of exercise. Measurements can be absolute values, changes in values over time periods, or other transformations of acquired data such as ratios, averages, or logarithms.

One important characteristic of biological data sets such as that of FIG. 1 is that the number of measurements n (also referred to as dimensions) is larger than the number of observations p, often by several orders of magnitude. It is not uncommon for hundreds or thousands of measurements to be acquired on samples from fewer than one hundred patients. Such a data set is referred to as a broad data set. In traditional machine learning applications, a large number of observations is typically available for training a classifier, and the data dimensionality is much smaller than the number of observations. Domain knowledge is often available to help pre-select the dimensions most relevant to the application. For biomarker discovery applications, it is often not possible to reduce the number of dimensions (measurements) based on domain knowledge. Currently, the biological processes underlying many diseases are still poorly understood. To study these diseases, it is necessary to measure and consider as many biological entities as possible, including those about which little is known. Existing data mining techniques either cannot be applied to broad data sets, or their accuracy is questionable under these conditions. As a result, new techniques are needed to extract biomarkers accurately.

A number of biomarker discovery methods have been proposed in the prior art. In general, these methods are not scalable to broad data sets with hundreds or thousands of measurements per observation, but apply only to data sets with dimensionality of a few hundred or fewer, and particularly to data sets having more observations that dimensions. For example, PCT publication number WO 01/44269 discloses novel brain protein markers indicative of a neurological disorder. 217 proteins were identified using two-dimensional gel electrophoresis, and a multivariate analysis revealed that eight of the proteins were related to one or more psychiatric diagnoses. In addition, a principle component analysis was performed to identify a panel of 19 proteins capable of distinguishing between normal and depression samples. While these techniques are useful for identifying important factors from a relatively small collection of potential biomarkers, they cannot be applied to a large number of measurements. When principle component analysis is applied to a data set of very high dimensionality, it may identify a small number of new dimensions most relevant for distinguishing classes. However, the new dimensions, which are linear combinations of the original dimensions, are not themselves measurable quantities. A large number of values must still be measured, and it is not practical for such a large number to be used as biomarkers. Thus the disclosed method cannot be used to discover biomarkers in broad data sets.

PCT publication number WO 00/70340 discloses a method for determining diagnostic markers indicative of particular types of cancer. Using two-dimensional gel electrophoresis, a large number of spots were identified from tumor cells and non-cancerous cells. Principle component analysis and partial least squares were applied to the variables to identify 170 markers capable of classifying samples into disease group. The set of markers discovered was only moderately successful, correctly classifying only 11 of 18 samples in the test set. In this method, the optimal number of markers desired is between 100 and 200. While this number is suitable for markers obtained from a single assay such as a two-dimensional gel, it is not very practical for measures obtained from a variety of different sources such as cytometers, mass spectrometers, and case report forms. Additionally, a prediction that is correct for only 61% of cases is not sufficiently accurate for most purposes. Furthermore, a model developed from such a small training set cannot be generalized reliably to unknown samples and therefore has little predictive accuracy. This method is therefore not suitable for discovering biomarkers in broad data sets containing data from a variety of sources.

A system for predicting future health is described in U.S. Pat. No. 6,059,724, issued to Campell et al. A set of biological measures is acquired from a large number of patients, each in one of two classes, and the measures are analyzed to locate biological markers capable of distinguishing between the classes. The number of measures to be considered is gradually reduced, and a discriminant analysis is performed on the remaining measures to identify a set of biological markers. The biomarkers can then be used to predict the risk of a new person of acquiring a disease corresponding to one of the classes. Although the method is stated to apply to any number of measures, the number of measures must be reduced sufficiently to allow the discriminant analysis to be performed; this analysis requires the number of measures to be smaller than the number of samples. In the example given, an initial set of 36 measures is reduced to 18 based on a sample size of over 400 patients. This is a qualitatively different problem from discovering biomarkers in an initial set of 5000, or even 1000, measurements from 100 subjects. Additionally, in this method, an important factor both in choosing the original set of potential biomarkers and in reducing the set is knowledge of the particular disease and of the biological factors already known to be important in the disease. This is almost the opposite of the problem of searching for markers not previously known to have any correlation with the disease of interest. The method produces a single set of biomarkers believed to distinguish the two classes, and the backward stepwise discriminant analysis employed does not allow for backtracking if an incorrect marker was removed from the set.

Similar problems have been addressed in the analysis of data produced by DNA microarrays, which provide expression data for thousands of genes in a single experiment. Most current approaches to the computational analysis of gene expression data attempt to learn functionally significant classifications of genes either in a supervised or unsupervised manner. Common techniques include hierarchical clustering, self-organizing maps, and support vector machines (SVM). In general, these techniques aim not to locate specific features capable of classifying patients, but rather to cluster different genes into functional classes. For example, hierarchical cluster analysis (HCA) has been used to visualize genes' functional relationships [M. B. Eisen et al., “Cluster analysis and display of genome-wide expression patterns,” Proc. Natl. Acad. Sci. 95, 14863-14868, 1998]. Based on the cluster trees obtained, a user can hypothesize new gene functional classes. SVMs have been used to classify genes based on gene expression, using a training set in which the number of genes (corresponding to observations) is larger than the number of dimensions (experiments) [M. P. S. Brown et al., “Knowledge-based analysis of microarray gene expression data by using support vector machines,” Proc. Natl. Acad. Sci. 97, 262-267, 20000]. When SVMs are applied to broad data sets, the resulting models are unreliable, i.e., not generalizable to unknown data beyond the training set. Additionally, SVMs are generally used to build a model from the entire data set, not from subsets of measurements within a data set.

Thus none of the prior art is suitable for discovering biomarkers within broad data sets, and there is still a need for a computationally efficient method of biomarker discovery in large volumes of high-dimensional biological data. There is a particular need for discovering biomarkers for diseases about which very little is known, where domain knowledge cannot be used to assist in the identification of relevant biomarkers.

### SUMMARY OF THE INVENTION

The present invention provides a method for identifying biological markers in broad data sets containing n biological measurements for each of p observations. The biological markers can be used to predict clinical endpoints, e.g., to classify observations into one of a number of clinical classes or to predict values of a continuous response variable such as disease severity. Preferably, n>10 p, and the measurements are obtained from different sources. Each biological marker consists of a group of at most k measurements; k is preferably less than p/5 and can be selected by a user or in dependence on a desired computation time or predictive accuracy. Thus the method is capable of efficiently locating small subsets of relevant biological measurements within large volumes of data. The method has two main steps: (a) reducing the set of n measurements to a set of m candidate measurements, and (b) selecting one or more biological markers (subsets of k or fewer measurements) from the set of m candidate measurements.

In one embodiment of the method, the set of n initial measurements is reduced by performing a correlation analysis, preferably a correlation-based cluster analysis, and most preferably a correlation-based hierarchical cluster analysis. The amount of reduction can depend upon a user-selected similarity threshold or on the reduction necessary to facilitate locating biomarkers with k or fewer members. Alternatively, or in addition to the correlation analysis, a differential significance analysis can be performed, in part in dependence on a user-selected hypothesis testing significance threshold.

Subsets of the measurements that serve as biological markers can be identified by examining all possible subsets of k or fewer measurements, preferably in parallel. Alternatively, the biomarkers can be found by non-exhaustive techniques such as simulated annealing. The identified biomarker subsets can then be ranked based on their accuracy of prediction. Additionally, a market-basket analysis can be performed on the identified biomarkers to locate recurring patterns of associations among measurements that make up the biomarkers.

The invention also provides a program storage device accessible by a processor and tangibly embodying a program of instructions executable by the processor to perform steps for the methods described above.

### BRIEF DESCRIPTION OF THE FIGURES

FIG. 1 is a table representing a broad data set of biological measurements of a number of observations, in which n>p.

FIG. 2 is a flow diagram of a biological marker discovery method according to the present invention.

FIG. 3 shows a correlation-based hierarchical cluster tree used in one step of the method of FIG. 2.

FIG. 4 is a flow diagram of a method for using the hierarchical cluster tree of FIG. 3 for variable reduction.

FIG. 5 is a block diagram of a scheme for parallel data mining for biological markers according to the method of FIG. 2.

FIG. 6 is a block diagram of a hardware architecture for implementing the scheme of FIG. 5.

FIG. 7 shows a sample space of biological markers containing at most three measures for use in a simulated annealing method to identify biological markers.

FIG. 8 is a flow diagram of a simulated annealing technique for use in the method of FIG. 2.

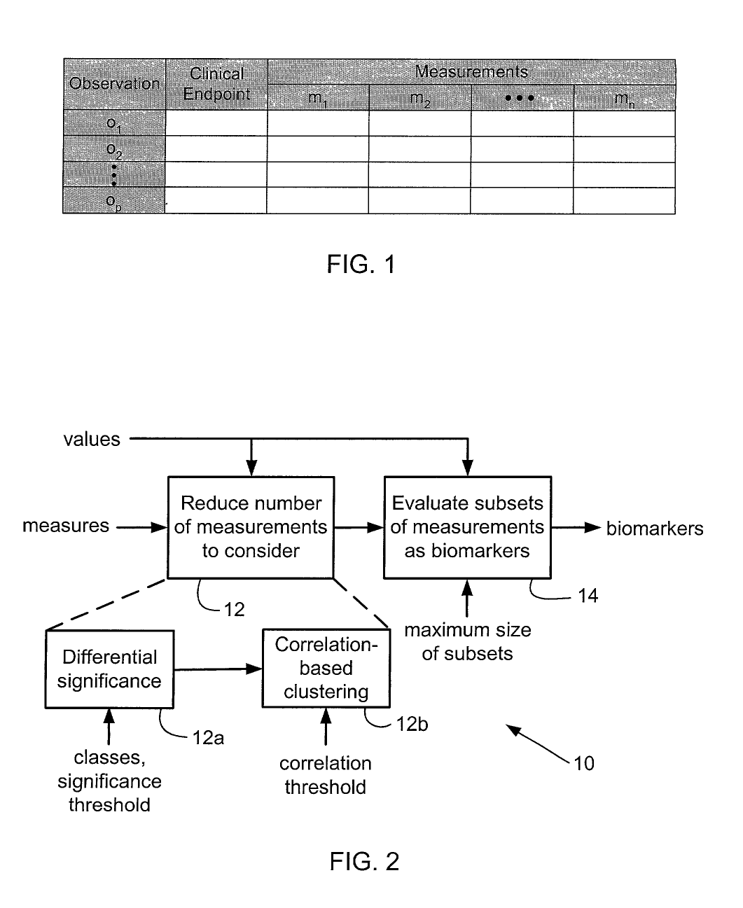

### DETAILED DESCRIPTION OF THE INVENTION

The present invention provides a method for mining broad biological data sets for biological markers that are predictive of a clinical endpoint. A clinical endpoint is a clinically meaningful measure of how a patient feels, functions, or survives. In general terms, there are two main types of predictive modeling involved, classification and regression. Classification predicts a subject's clinical class such as disease condition, response to therapy, or other categorical clinical endpoints. Any conceivable classification for which biological markers are desired is within the scope of the present invention. Regression predicts the value of a clinically-relevant continuous variable such as disease severity or progression.

In broad data sets, the number of measurements or dimensions n is much larger than the number of observations p (e.g., biological samples or subjects in an experimental study). In a preferred embodiment, n>10 p. Measurements can include any quantitative or qualitative (categorical) biological factors; examples include but are not limited to blood cell populations, cell-surface antigen levels, and soluble factor concentrations obtained from cytometry measurements; levels of specific proteins or small organic molecules in tissue or biological fluids; gene expression data from DNA microarray hybridization experiments; spectral components generated by techniques such as mass spectrometry or chromatography (e.g., mass spectrum peaks); concentrations of molecules obtained from immunoassays; responses to health-related questionnaires; and patient data obtained from case report forms. It is not uncommon for between five and ten thousand measurements to be acquired for each of fewer than one hundred subjects. For the purposes of the present invention, the source and nature of the biological measurements are irrelevant. Preferably, however, measurements are obtained from a variety of different sources and mined together.

Rather than consider each measurement as a potential biological marker, as is commonly done in the prior art, the present invention considers a biological marker to be a set of measurements, i.e., a subset of the total number of measurements. Typical subset sizes are less than ten. In addition, the present invention considers that there are multiple biomarkers for predicting a given clinical endpoint, and that different biomarkers can include different numbers of measures. For example, the two best biomarkers for a particular disease can be a set of six biological measurements and a set of three biological measurements. These different measurement sets may have overlapping members. The maximum number of measures k in a biomarker is preferably less than the number of observations p. In a preferred embodiment, k<p/5, and most preferably, k<p/10. Note that these restrictions are somewhat arbitrary; the reasons for limiting k are to reduce the number of measurement subsets that are potential biomarkers, and to limit the number of measurements that must be obtained once a biomarker has been established. Large numbers of measurements are not practical for inclusion in biomarkers.

In contrast to prior art measurements used as biomarkers, measurements in the biomarkers of the present invention preferably include those at much lower granularity. For example, rather than concentrations of blood cell populations such as CD4⁺ T cells, measurements of the present invention can include subspecies of CD4⁺ T cells. One reason for considering lower-grained factors is that modem bioanalytical instruments are capable of making such fine-grained measurements. Clearly, if finer grained measurements are being obtained, then a larger number of total measurements is produced and considered for inclusion in biomarker sets. Additionally, the biomarkers of the present invention can include measurements that do not correspond directly to known biological entities. For example, features of spectral data can include peak locations (i.e., mass-to-charge ratios) and intensities whose responsible molecular species are not yet determined.

In addition, because of the potential interactions between biological entities, many of which are currently unknown, derived measures are commonly considered in addition to base measures. A base measure is one that is acquired directly, while a derived measure is obtained by combining or otherwise transforming base measures. For example, the ratio between T cell and total white blood cell count is known to be a better indicator of asthma that either absolute cell count by itself.

Allowing for such combinations of an already large number of potential measurements increases the number of measurements to consider enormously.

Note that there are two types of values associated with each observation, and that the distinction between the two is somewhat arbitrary. One type is measurements, values that are measured using bioanalytical instruments. The other type, referred to as annotations, can include any descriptor not having a value obtained from an analytical instrument. For example, the class to which each subject belongs (disease versus not disease, drug responder versus non-responder) is a descriptor. Subject data such as age, sex, and lifestyle information can be either measurements or annotations. External factors (e.g., pollen count for allergy treatment studies) are also relevant annotations. When used as measurements, these data are treated just as bioanalytical measurements are. However, when used as annotations, the values can serve as additional factors for defining a response variable whose value is predicted, e.g., female drug responders versus female non-responders.

Given this framework, choosing subsets of at most k measurements from an initial set of n measurements, the total number of possible biomarkers is ₙCₖ+ₙCₖ₋₁+ . . . +ₙCₗ. Using standard notation, ₙCₖ represents the number of distinct combinations of k objects from a set of n objects. For a typical data set, figures are as follows:

| | |
|---|---|
| Number of variables (measurements) | 5000 |
| Number of available samples | 200 |
| Maximum size of biomarker to consider | 40 |
| Number of potential biomarkers | >10⁸⁰ |

Clearly, when the number of variables is large, it is not feasible to examine systematically all potential subsets of measurements. The high combinatorics involved in mining broad data sets makes it imperative to reduce the number of variables from which biological markers can be derived.

A flow diagram outlining the general steps of a method 10 of the invention is shown in FIG. 2. Inputs to the method 10 are the measurements and their values, and the method outputs a set of one or more biomarkers. As shown, the method has two broad steps, reducing the number of potential measurements to include in the biomarkers (step 12), and identifying subsets of measurements to serve as biomarkers (step 14). The amount of reduction in step 12 depends upon a variety of factors including user-specified thresholds, the maximum number k of measures to include in a biomarker, the number of observations p and initial measurements n, and processing and time constraints. Individual steps and specific implementation methods are described below for performing the two main method steps. Although the method can be implemented with all of the individual steps performed sequentially, it can also be performed with only a few of the individual steps. The step order can also be varied as desired.

The first step 12, dimensionality reduction, assumes that among the initially large pool of dimensions, many are not useful in discriminating between different clinical classes or predicting response variable values and thus can be eliminated from consideration. Preferably, two types of dimensionality reduction steps are included. One type of dimension to eliminate is an irrelevant dimension, i.e., one that cannot by itself predict a clinical endpoint. In step 12a, referred to as differential significance evaluation, each dimension is evaluated separately, using any technique that scores how well it can discriminate between classes or predict the response variable. Dimensions that are not sufficiently effective at predicting, as defined by a user-selected significance threshold, are eliminated from consideration.

In the case of classification, for each measurement, the mean values of the different clinical classes are compared to determine whether they are statistically significantly different. Any statistical method that tests for significant difference between independent sample populations can be used. One suitable method is the non-parametric Kruskal-Wallis test, which makes no assumption about data distribution. Alternatively, for normally distributed data, the ANOVA F-statistic can be used. In any method, dimensions are eliminated based on a threshold p-value, which can be set by the user. The p-value indicates the probability that the mean values could have been identical by chance alone. P-values can be adjusted to correct for multiple tests being performed on a single data set, using, e.g., a Bonferroni or Bayesian correction. A typical threshold p-value is 0.05, but values as low as 0.001 can be used. Dimensions yielding p-values exceeding the threshold can be eliminated from consideration for inclusion in biomarker sets. For regression, each measurement is correlated with the continuous outcome variable. A low correlation eliminates the measurement from further consideration. The user can select a p-value or correlation coefficient threshold to determine whether a measurement will be eliminated.

The second type of variable to eliminate is a redundant variable, one that is strongly similar to another variable and therefore provides no additional information. All variables that are sufficiently similar can be replaced by any one of them. In step 12b, a correlation analysis is performed to determine sets of variables that are sufficiently similar to be considered redundant. Note that unlike step 12a, which is specific to the clinical endpoint considered, similarity between variables is independent of class or response variable. A measure of correlation such as a Pearson (parametric) or Spearman (non-parametric) correlation test is used to evaluate variable similarity. Any pair or group of variables whose similarity exceeds a user-specified similarity or correlation threshold can be replaced by one of the variables in the group, with the rest eliminated from consideration. Preferably, the most relevant variable of the group, as determined by its differential significance, is retained.

In addition to simply reducing the number of relevant variables to consider, the correlation step 12b helps improve the success of the linear predictive models developed in the subsequent step 14. In such models, highly correlated variables generate nearly singular matrices that are problematic for many algorithms to invert. Furthermore, when linear model coefficients are used to assess the importance of associated variables, coefficients of highly correlated variables are divided among variables, resulting in an artificially decreased apparent importance of the variables.

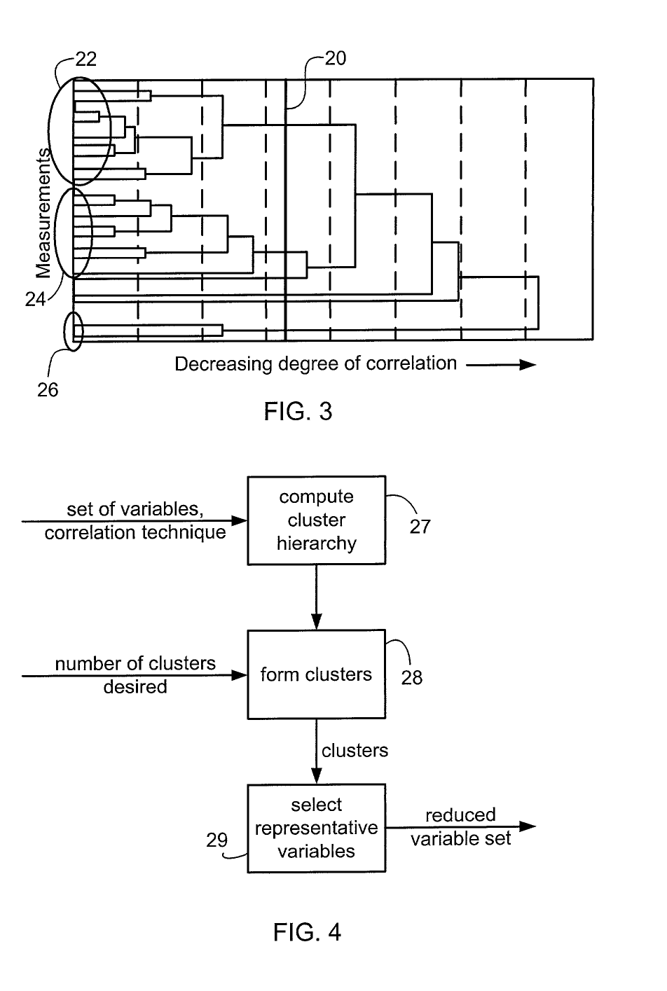

In a preferred embodiment of the method 10, the correlation analysis 12b is a correlation-based hierarchical cluster analysis (HCA). HCA is a well-known technique, but to the knowledge of the present inventor, has never been applied to dimensionality reduction for biological data mining. This technique is illustrated in FIG. 3, a hierarchical cluster tree of a set of variables, in which variables are clustered at various levels of similarity. Variables are compared using one of a number of correlation measures such as Pearson or Spearman. Any suitable linkage rule can be used for creating clusters of clusters. Preferably, the linkage rule is complete linkage, which ensures that any two points within the cluster satisfy the correlation threshold. The horizontal axis of the diagram represents decreasing correlation of measurements or variables within the clusters.

For the present invention, the variable reduction can be performed in one of two ways. In one method, a threshold correlation value is selected on the horizontal (correlation) axis. Variables contained within the same cluster to the left of this threshold, shown as a line 20 in FIG. 3, are considered to be interchangeable and therefore redundant. That is, they all provide the same information for predicting the clinical endpoint. One variable from each such cluster is retained for consideration, while the others are eliminated. For example, in FIG. 3, each of the clusters 22, 24, and 26 is replaced by a single variable. Alternatively, as shown in the flow diagram of FIG. 4, the degree of variable reduction, i.e., the number of clusters desired, can be selected by the user based on computing bandwidth and time constraints, and the similarity threshold chosen to achieve the desired reduction. In this method, given as input a set of variables and a correlation technique, a cluster hierarchy is developed in step 27. Next, based on the number of clusters desired, which can be user selected, the clusters are formed in step 28. Because one measurement is retained from each cluster, the number of clusters desired is equal to m, the number of candidate measurements remaining after step 12. A representative measurement is chosen from each cluster in step 29, e.g., the measurement with the highest statistical significance in differentiating among classes. The reduced variable set is then returned.

Note that the user-selected thresholds for steps 12a and 12b have a significant effect on the resulting sets of biomarkers. If the data reduction is too aggressive, then information is lost and good biomarkers might not be discovered. This can occur particularly for dimensions that are bad predictors individually but excellent predictors when used in combination with other variables. However, if the data are not reduced sufficiently, then step 14 (described below) will be too computationally intensive to arrive at the biomarkers efficiently.

The user-selected thresholds can be derived based on a desired computation time. For example, the amount of time necessary to perform the subsequent step 14 can be determined empirically for a variety of data set sizes. In general, a formula for computation time cannot be determined, because of unknown processor-dependent factors, but the time can be determined empirically. The user can then select a desired computation time, and the required data reduction can be determined from the empirical results. The necessary data reduction determines the number of clusters m to select, which is an input to step 28 of FIG. 4.

After the number of variables is reduced sufficiently, step 14, selection and evaluation of subsets of measurements as biomarkers, is performed. The user can select a value of k, the maximum size of the subsets, as input to step 14. Broadly, there are two types of subset selection, an exhaustive search method and a heuristic method that finds a few good but not necessarily globally optimal biomarkers.

In the first type of method, an exhaustive search is used to find globally optimal biomarkers. Typically, the exhaustive search is best performed when step 12 has yielded sufficient dimensionality reduction. For example, a suitable scenario is as follows:

| | |
|---|---|
| Original number of measurements | 5000 |
| Number of observations | 150 |
| Maximum number of measurements per biomarker subset | 5 |
| Number of measurements after differential significance step | 100 |
| Number of measurements after correlation step | 35 |
| Number of potential biomarkers = ₃₅C₅ + ₃₅C₄ + ₃₅C₃ + | <10⁶ |
| ₃₅C₂ + ₃₅C₁ | |

When the number of potential biomarkers is small enough, it is computationally feasible to enumerate and evaluate each potential biomarker. In this process, all subsets of between one and k variables are enumerated from the measurements remaining after the final dimension reduction step. For each such subset, a test is applied to determine the subset's accuracy at predicting classification or response variable values. For example, a discriminant analysis can be used. In some cases, it may be desirable to begin evaluating subsets of 1 or 2 measurements and then proceed to subsets of increasing size until subsets of k measurements are evaluated. In these situations, the measurement pairs with low predictive accuracy can be eliminated from consideration in larger subsets, particularly when available computation time is limited. For example, consider the case of m=100 and k=5. Subsets of size 1, 2, and 3 can be evaluated relatively quickly. For subsets of size 4, ₁₀₀C₄ is approximately 4×10⁶, which can still be computed in a reasonable amount of time. ₁₀₀C₅, however, is approximately 76×10⁶, which (at current processor speeds) is not feasible to compute in a reasonable amount of time with a reasonable number of processors. By keeping only a small number of the best 4-tuples, however, the number of measurements to consider for inclusion in 5-tuples can be reduced, e.g., to 50. Then ₅₀C₅, which is less than 3×10⁶, is more manageable to compute.

Accuracy can be determined by any suitable error measurement. For example, classification accuracy can be assessed as the percentage of correct classifications. In the case of two classes such as disease and not disease, the error rate can be reported as the numbers of false positives, i.e., samples incorrectly classified into the disease group, and false negatives, disease samples classified as not diseased. In general, because false positives and false negatives are related, a higher false positive rate is preferred to minimize the number of false negatives, but the desired ratio depends on the particular data set. To measure predictive accuracy of regression, any suitable fitness criterion, such as the adjusted R² criterion, can be used. After evaluation, subsets are ranked by accuracy, and the top few subsets selected to be biomarkers. To better estimate predictive accuracy, a technique such as cross validation, leave-one-out, or bootstrapping is preferably used.

Because each potential biomarker can be evaluated independently, the evaluation is preferably parallelized. In a parallel process, different portions of the potential biomarker space are evaluated by different processors to reduce the total time to evaluate all biomarkers. In many cases, the ability for parallel biomarker evaluation enables an exhaustive search that would be prohibitively slow if only a single processor were used.

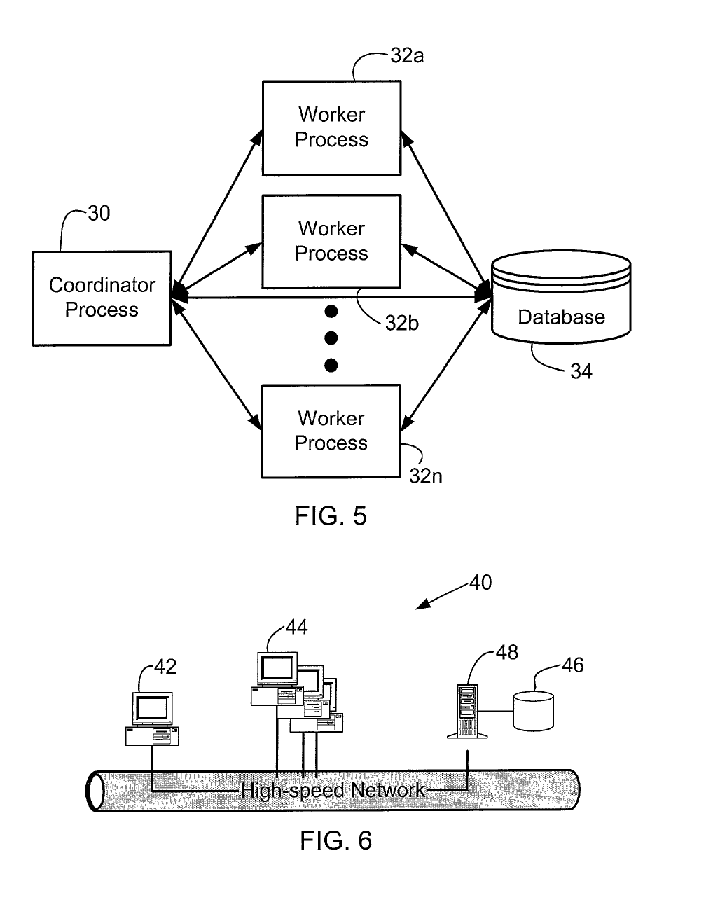

A suitable scheme for parallel biomarker evaluation is shown in the block diagram of FIG. 5. In this scheme, a coordinator process 30 coordinates biomarker evaluation performed by any number of worker processes 32a through 32n. Each worker process 32 evaluates a different portion of the potential biomarker space. In one possible implementation, the coordinator process 30 maintains three lists of biomarkers: one of biomarkers that have already been evaluated, one of biomarkers that are currently being evaluated, and one of biomarkers that are yet to be evaluated. The coordinator process selects a subset of potential biomarkers from the third list, selects a free worker process 32, and sends the subset to the worker process 32. The worker process 32 uses the received instructions to download from a database 34 all data required for evaluating the biomarker. Upon completion of the evaluation, the worker process 32 sends the results of the evaluation to the coordinator process 30, which updates its three lists accordingly. The coordinator process 30 then saves the evaluation results to the database 34. When all biomarkers have been evaluated, the coordinator process 30 sorts the biomarkers based on the evaluation results and returns the best ones.

This implementation can be made very efficient with the proper choice of representation for potential biomarkers. For small values of m, one technique is to use a bitmap representation, in which each potential biomarker subset is represented by a binary number, each position of which corresponds to a particular measurement. A 1 in the position means that the measurement is included in the potential biomarker, and a 0 means it is not. A given biomarker then contains all the measurements whose corresponding positions contain 1's. Each subset is uniquely defined by the integer of its binary representation, and the entire set of biomarkers is enumerated simply by counting from one to the maximum number of potential biomarkers. To represent the three lists described above, it is necessary only to maintain a current count C, the maximum integer value of biomarkers already evaluated or currently being evaluated, and a small list of the biomarkers currently being evaluated. As will be apparent to those of skill in the art, there are numerous efficient biomarker representations for larger values of m.

A hardware system 40 for implementing the parallel exhaustive biomarker search is shown in FIG. 6. The system 40 corresponds to a typical networked personal computer system that exists in most corporate environments or a dedicated high-performance, low-cost compute cluster. One workstation 42 acts as the coordinator and initiates and manages biomarker evaluation. A subset of or all of the remaining workstations 44 accessible from the network form the worker processors. A database server 48 controls access to the database 46 that stores potential biomarkers and other relevant data. For example, the coordinator workstation 42 can use NT lightweight threads and each workstation 44 can run a DCOM-interface biomarker evaluation procedure.

In an alternative embodiment of the exhaustive search method, the complete biomarker space is not searched. This may be necessary if there are too many potential biomarkers or if the user desires to impose arbitrary computational resource limitations, such as response time or percentage of the biomarker space searched. In this case, a sorted list is maintained of the biomarkers that have already been evaluated, and the process can be stopped at any time and the current best biomarkers extracted. Preferably, the coordinator process can stop the search and resume where it left off. When the coordinator process receives a signal to stop the search, it stops assigning new tasks to the worker processes and waits to receive current evaluation results from the ongoing worker processes. It then saves the value of C, the highest biomarker that has been evaluated, to allow resumption of the evaluation process. In order to allow the user to stop the process at any point, a computation thread is added to the coordinator process to detect events from the user interface.

In the second type of biomarker selection method, the complete biomarker space is not searched exhaustively. Rather, a heuristic technique is used that finds a few good, but not necessarily globally optimal, solutions. In general, any existing technique for feature subset selection can be used in the context of biomarker discovery according to the present invention. Feature subset selection methods typically find one good subset of the data, but can also be used to find multiple good subsets.

One suitable technique is simulated annealing, a method used in large optimization problems to find solutions that are good but not necessarily globally optimal. For example, simulated annealing has been used extensively for layout problems in circuit design. It has also been used in the field of chemometrics for determining three-dimensional molecular structure information to predict the toxicity of novel compounds produced by combinatorial chemistry. However, it has not previously been applied to biomarker discovery. The method is analogous to heating a crystalline material and then slowly cooling it, causing it to anneal. During the slow cooling, the molecules of the material can move around and settle into lower energy states. In the biomarker discovery context, multiple iterations of random changes are made to an initial biomarker, and the changes are either accepted or rejected. Higher energy states are analogous to less accurate biomarkers; there is always some probability that changes to higher energy states will be accepted. Changes to more accurate states are always accepted. The method begins at one “temperature,” and the temperature is decreased in stages. As the temperature decreases, it is less likely that changes to higher energy states will be accepted. Thus the search is much more likely to backtrack during the initial stages.

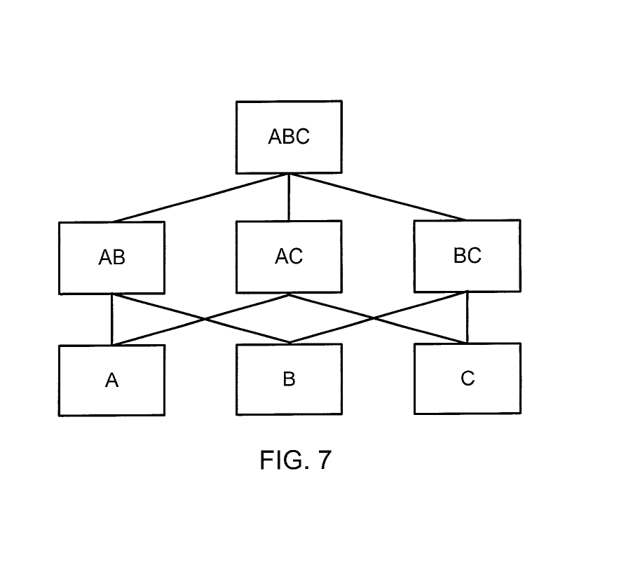

The states of the biomarker search space consist of sets of measurements containing k or fewer members. A state change represents either adding a measurement to or removing a measurement from a given state. For example, consider a search for biomarkers containing three or fewer measures from a set of measures A, B, and C. FIG. 7 illustrates the biomarker search space containing seven potential biomarkers. Lines connect states that differ by the addition or removal of a single measure. For example, state AC has three possible next states, ABC, A, and C. State AC can be changed to state A by removing measure C or state C by removing measure A. State AC cannot be changed directly to state BC, but can be changed first to C and then to BC. This representation of the biomarker search space satisfies the ergodicity property required for simulated annealing: any biomarker can be changed directly or indirectly to any other biomarker by successively adding or removing variables.

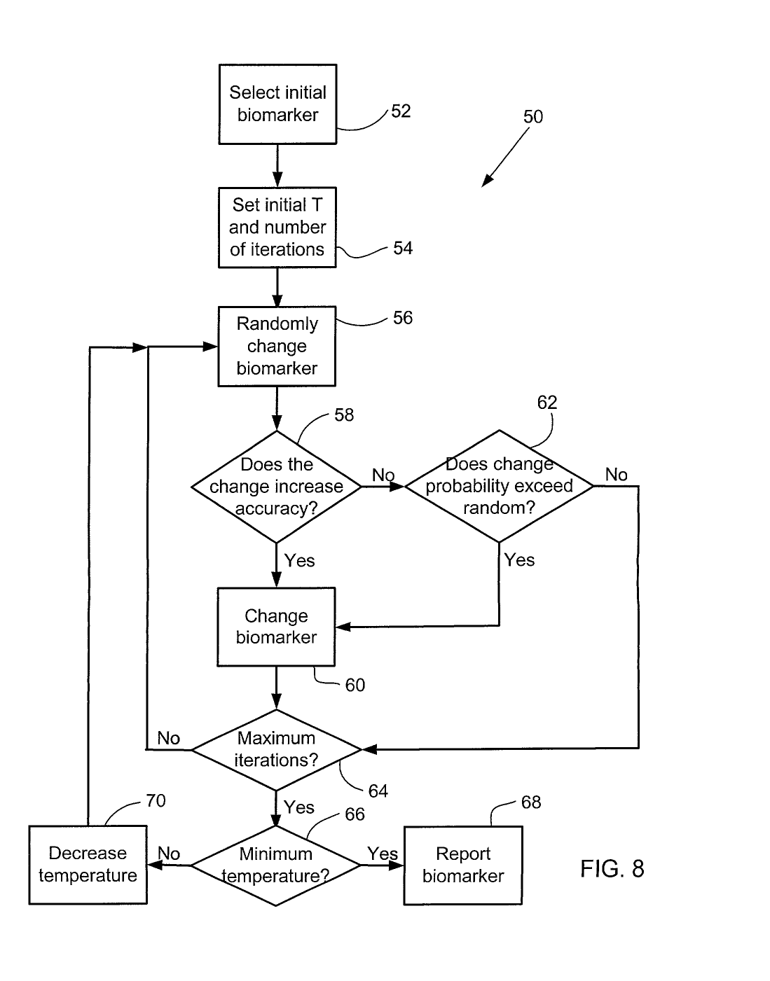

A flow diagram of a simulated annealing method 50 for searching for biomarkers is shown in FIG. 8. In a first step 52, a potential biomarker is selected randomly as a set of k or fewer measurements. An initial “temperature” T and number of iterations per stage are selected in step 54, as well as the amount of temperature decrease in each stage. T can be thought of as a parameter that controls how much the method relies upon randomization. A single random change is made to the potential biomarker in step 56 by adding or removing a measurement. The accuracy of each biomarker in predicting endpoints is then evaluated, e.g., by discriminant analysis. The accuracies Aᵢ of the original (1) and changed (2) biomarkers are compared in step 58. If the accuracy improves, the change to the new biomarker is made (step 60). However, if the accuracy does not improve, the following probability (Boltzmann factor) is evaluated in step 62: e^{(A}^{₂}^{-A}^{₁}^{)/T}. The change to a less accurate biomarker is made based on this probability (e.g., by comparing it to a randomly generated number between 0 and 1), with the method passing to step 60 if the change is made and step 64 if not. Including changes to higher energy states prevents the system from getting stuck in local energy minima. Note that changes to higher energy states are more likely to occur at high temperatures than at low temperatures.

The method next evaluates whether the maximum number of iterations per temperature stage (step 64) has been reached. If not, the method returns to step 56 to make a random change to the current biomarker. If the maximum number has been reached, the temperature is evaluated in step 66 to determine whether the minimum temperature has been reached. If it has, the method ends at step 68 and the current biomarker is reported. Alternatively, the temperature is lowered in step 70 and the method returns to step 56.

A variety of parameters must be set upon beginning the simulated annealing method. Any suitable values for the initial temperature, temperature decrease per stage, and number of iterations per stage can be used; optimal values typically depend upon the data set. Preferably, the number of iterations per stage is chosen so that the most accurate biomarker is found at each temperature. One way to select the initial value of T is to begin with a value of 1 and successively double the value until an acceptance rate of 90% is achieved in 100 possible random changes. T can then be reduced linearly, e.g., T_{new}=αT_{old}, with α between 0 and 1. Typically, the optimal parameters can be determined empirically.

Simulated annealing arrives at a single good biomarker made up of k or fewer measurements. However, the method can also be used to obtain multiple biomarkers. Because simulated annealing is a probabilistic method, it does not produce the same result when repeated. Thus the simulated annealing algorithm can be run as many times as the number of desired biomarkers, each time producing a different measurement subset.

Biomarkers identified by the method of the invention are used to predict clinical endpoints of new observations, such as clinical classifications or response variable values. Measurements are taken of the variables in the final biomarker set, and their values used to determine a value of the response variable or in which class the subject falls.

An optional additional step can be included after a number of biomarker subsets have been selected by any of the above-listed or other exhaustive or non-exhaustive search methods. In this additional step, a market-basket analysis is performed to identify patterns of recurring subsets of measurements among identified biomarkers. Each biomarker is treated as a market basket, with measurements analogous to items in the basket. Any existing method for association rule mining can be used. One suitable algorithm is the well-known Apriori algorithm [R. Agrawal et al., “Fast Algorithms for Mining Association Rules,” Proc. 20th Int. Conf. Very Large Data Bases, 487-499, 1994]. The market-basket analysis can be performed on a predetermined number of the highest-ranked biomarkers or on all biomarkers exceeding a user-set accuracy threshold. The resulting frequent itemsets represent combinations of measurements that occur frequently in good biomarkers, allowing the user to gain biological insight into how certain combinations of measures are correlated with clinical outcomes.

The goal of the market-basket analysis is not primarily to determine the most important measurements to predict a particular clinical endpoint, but rather to gain biological insight into a medical condition or drug activity. For example, if a high value of measurement X often occurs with a low value of measurement Y, then these measurements might indicate previously unknown pathogenic mechanisms. The results of the market-basket analysis can then be used to direct further biological research.

Although not limited to any particular hardware configuration, the invention is preferably implemented in one or more computers, each containing a processor, data storage device, memory, and input and output devices. The data set is stored in a database accessed by the computer. Methods of the invention are executed by the processor under the direction of computer program code stored within the computer. Using techniques well known in the computer arts, such code is tangibly embodied within a computer program storage device accessible by the processor, e.g., within system memory or on a computer readable storage medium such as a hard disk or CD-ROM. The methods may be implemented by any means known in the art. For example, any number of computer programming languages, such as Java, C++, or LISP may be used. Furthermore, various programming approaches such as procedural or object oriented may be employed. It is to be understood that the steps described above are highly simplified versions of the actual processing performed by the computers, and that methods containing additional steps or rearrangement of the steps described are within the scope of the present invention.

It should be noted that the foregoing description is only illustrative of the invention. Various alternatives and modifications can be devised by those skilled in the art without departing from the invention. Accordingly, the present invention is intended to embrace all such alternatives, modifications and variances which fall within the scope of the disclosed invention.

What is claimed is:

1. A method for identifying biological markers in a set of n biological measurements for each of p observations, wherein n>p and each observation is associated with a clinical endpoint, each biological marker comprising at most k measurements, wherein k<p, said method comprising:
a) reducing said set of n measurements to a set of m candidate measurements; and
b) selecting at least two biological markers from said set of m candidate measurements, wherein values of each biological marker predict said clinical endpoints.

2. The method of claim 1, wherein said clinical endpoints correspond to clinical classes.

3. The method of claim 1, wherein said clinical endpoints correspond to a continuous response variable.

4. The method of claim 1, wherein n>10 p.

5. The method of claim 1, wherein k<p/5.

6. The method of claim 1, wherein step (a) comprises performing a correlation analysis.

7. The method of claim 6, wherein said correlation analysis comprises a correlation-based cluster analysis.

8. The method of claim 7, wherein said correlation-based cluster analysis comprises a correlation-based hierarchical cluster analysis.

9. The method of claim 6, wherein said correlation analysis is performed in part in dependence on a user-selected correlation threshold.

10. The method of claim 6, wherein said correlation analysis is performed in part in dependence on a user-selected value of m.

11. The method of claim 1, wherein step (a) comprises performing a differential significance analysis.

12. The method of claim 11, wherein said differential significance analysis 15 is performed in part in dependence on a user-selected significance threshold.

13. The method of claim 1, wherein said n measurements have different sources.

14. The method of claim 1, further comprising ranking said selected biological markers.

15. The method of claim 14, wherein said biological markers are ranked in dependence on an accuracy of predicting said clinical endpoints.

16. The method of claim 1, wherein said biological markers are selected from all possible subsets of at most k measurements of said set of m measurements.

17. The method of claim 16, wherein said biological markers are selected by evaluating each of said possible subsets.

18. The method of claim 17, wherein said possible subsets are evaluated in parallel.

19. The method of claim 1, wherein step (b) comprises simulated annealing.

20. The method of claim 1, wherein k is a user-selected value.

21. The method of claim 1, wherein k is selected in dependence on a desired computation time.

22. The method of claim 1, wherein m is selected in dependence on a desired computation time.

23. The method of claim 1, further comprising performing a market-basket analysis of said selected biological markers.

24. A method for identifying a biological marker in a set of n biological measurements for each of p observations, wherein n>p and each observation is associated with a clinical endpoint, each biological marker comprising at most k measurements, wherein k<p, said method comprising:
a) reducing said set of n measurements to a set of m candidate measurements; and
b) using simulated annealing, selecting a biological marker from said set of m candidate measurements, wherein values of said biological marker predict said clinical endpoints.

25. The method of claim 24, wherein n>10 p.

26. The method of claim 24, wherein k<p/5.

27. The method of claim 24, wherein step (a) comprises performing a correlation analysis.

28. The method of claim 27, wherein said correlation analysis comprises a correlation-based cluster analysis.

29. The method of claim 28, wherein said correlation-based cluster analysis comprises a correlation-based hierarchical cluster analysis.

30. The method of claim 27, wherein said correlation analysis is performed in part in dependence on a user-selected correlation threshold.

31. The method of claim 27, wherein said correlation analysis is performed in part in dependence on a user-selected value of m.

32. The method of claim 24, wherein step (a) comprises performing a differential significance analysis.

33. The method of claim 32, wherein said differential significance analysis is performed in part in dependence on a user-selected significance threshold.

34. The method of claim 24, wherein said n measurements have different sources.

35. The method of claim 24, wherein k is a user-selected value.

36. The method of claim 24, wherein k is selected in dependence on a desired computation time.

37. The method of claim 24, wherein m is selected in dependence on a desired computation time.

38. The method of claim 24, further comprising performing a market-basket analysis on said selected biological markers.

39. A method for identifying at least one biological marker in a set of n biological measurements for each of p observations, wherein n >1 Op and each observation is associated with a clinical endpoint, each biological marker comprising at most k measurements, wherein k <p, said method comprising:
a) reducing said set of n measurements to a set of m candidate measurements; and
b) selecting at least one biological marker from said set of m candidate measurements, wherein values of each biological marker predict said clinical endpoints.

40. A program storage device accessible by a processor, tangibly embodying a program of instructions executable by said processor to perform method steps for a biological marker identification method, wherein said method identifies biological markers in a set of n biological measurements for each of p observations, wherein n>p and each observation is associated with a clinical endpoint, each biological marker comprising at most k measurements, wherein k <p, said method steps comprising:
a) reducing said set of n measurements to a set of m candidate measurements; and
b) selecting at least two biological markers from said set of m candidate measurements, wherein values of each biological marker predict said clinical endpoints.

41. A program storage device accessible by a processor, tangibly embodying a program of instructions executable by said processor to perform method steps for a biological marker identification method, wherein said method identifies a biological marker in a set of n biological measurements for each of p observations, wherein n>p and each observation is associated with a clinical endpoint, each biological marker comprising at most k measurements, wherein k <p, said method steps comprising:
a) reducing said set of n measurements to a set of m candidate measurements; and
b) using simulated annealing, selecting a biological marker from said set of m candidate measurements, wherein values of said biological marker predict said clinical endpoints.

42. A program storage device accessible by a processor, tangibly embodying a program of instructions executable by said processor to perform method steps for a biological marker identification method, wherein said method identifies at least one biological marker in a set of n biological measurements for each of p observations, wherein n>10 p and each observation is associated with a clinical endpoint, each biological marker comprising at most k measurements, wherein k<p, said method steps comprising:
a) reducing said set of n measurements to a set of m candidate measurements; and
b) selecting at least one biological marker from said set of m candidate measurements, wherein values of each biological marker predict said clinical endpoints.
